(19) United States
(12) Patent Application Publication
(10) Pub. No.: US 2015/0297345 A1
(43) Pub. Date: Oct. 22, 2015

(54) Insertion Tool for a Heart Valve

(71) Applicant: DP SHAW LIMITED, Christchurch (NZ)

(72) Inventor: David Peter SHAW, Christchurch (NZ)

(21) Appl. No.: 14/440,853

(22) PCT Filed: Nov. 7, 2013
(86) PCT No.: PCT/IB2013/059962

§ 371 Date: May 5, 2015

(30) Foreign Application Priority Data
Nov. 9, 2012 (NZ) 603501
Mar. 19, 2013 (NZ) 608426

(51) Int. Cl. A61F 2/24

(52) U.S. Cl. CPC A61F 2/2427

(57) ABSTRACT

An insertion tool for a heart valve includes a solid body portion in a first configuration having a first size and shape. The solid body portion in a second configuration having a second size smaller than the first size; said solid body portion comprises two or more parts; in the first configuration, the solid body portion provides a support portion for supporting a valve to be inserted by the insertion tool, and an insertion surface which protrudes distally from the support portion; the insertion surface is tapered from a small cross-section distally of the support portion to a greater cross section proximally to the support portion; in use the insertion surface elastically expands the periphery of an annulus into which the valve is to be fitted.

### TECHNICAL FIELD

The present invention relates to a tool for inserting a heart valve into a patient, and to a method of use of this tool.

### BACKGROUND ART

When a damaged heart valve is replaced by a prosthetic valve, the damaged valve is excised by cutting away the damaged leaflets together with any damaged material in the annulus upon which the natural leaflets are supported. This leaves the annulus prepared to support the prosthetic valve, which must then be secured in position in or on the annulus in such a manner that there is no significant leakage of blood between the margin of the prosthetic valve and the surrounding annulus.

A range of different designs have been used to overcome this problem—the earliest solution was to form a valve with a cylindrical housing which was secured in the aorta using external clips around the aorta (the Huffnagle valve).

Prior to the development of contemporary techniques for myocardial preservation, early valve replacements had to be carried out without any myocardial protection so that speed was essential—to facilitate this, several designs of heart valve were proposed with hooks around the perimeter of the valve (for example the McGovern Cromie Valve). The hooks were designed to bite into the surrounding annulus to give rapid securement. Unfortunately, designs of this type all had problems with achieving an adequate seal between the annulus and the valve perimeter, leading to an unacceptably high incidence of para-valvular leaks.

Once techniques for myocardial preservation were developed, more time was available for the installation of the prosthetic valve, and a number of prosthetic valve designs were produced with a sewing cuff made of a suitable fabric around the perimeter of the valve, so that the valve could be sutured to the surrounding annulus. Installing a valve of this type is relatively time-consuming, but gives good results if fitted properly.

A further range of recent prosthetic heart valve designs has sought to provide suture-less valves, i.e. valves which can be fitted without the need for suturing the valve in place. These valves include a range of different designs, but all include some means for partially collapsing the valve, inserting the valve into position in the annulus, and then expanding the valve against the annulus (using a variety of different mechanisms) to achieve a good fit. Examples of this type of valve and fitting system are the Edwards Odyssey/Intuity Valve and the Sorin PERCEVAL (trade mark) Valve.

U.S. Pat. No. 8,308,798 discloses a system for fitting a valve where, as a first step, an expandable stent is fitted into the annulus, expanded by means of a balloon catheter, and a valve is then fitted into the expanded stent. This technique, aspects of which also are disclosed in related US patents 2010/0131039 and U.S. Pat. No. 8,348,998, was developed to reduce the insertion time required for accurate insertion of a valve, but is not designed for use with rigid valves.

There have also been proposed designs for valve holders which do not require the valve to be partially collapsed; two examples of this type of the holder are described in U.S. Pat. Nos. 5,824,068 and 6,019,790. However, neither of these holders provides support for the valve uniformly around the whole of the portion of the valve which in use fits into the annulus, nor do they provide any means for assisting with the introduction of the valve into the annulus.

### DISCLOSURE OF INVENTION

An object of the present invention is the provision of an insertion tool for a heart valve which overcomes at least some of the above-mentioned drawbacks.

The insertion tool of the present invention has been developed for installing the heart valve described in New Zealand Patent No. 527025, and will be described with particular reference to that application. However, it is envisaged that the tool of the present invention could be used to insert any of a wide range of different designs of prosthetic heart valve.

As used herein, the terms “proximal/proximally”, “distal/distally” are used from the reference point of a surgeon using the equipment, i.e. “proximal/proximally” means that the component referred to is closer to the surgeon and “distal/distally” means that the component referred to is further away from the surgeon.

The present invention provides an insertion tool for a heart valve, said tool including a solid body portion which provides a surface of a first size and shape in a first configuration, and which forms two or more parts each of which is of a lesser size than said first size and shape, in a second configuration;

wherein, in said first configuration said body portion provides:
a support portion for supporting thereon a valve to be inserted by said tool; and
an insertion surface arranged to protrude distally from said support portion;
   and wherein said insertion surface is tapered from a small cross-sectional size distally of said support portion to a greater cross-sectional size proximally to said support portion, such that in use said insertion surface can gently and elastically expand the periphery of an annulus into which said valve is to be fitted, as said insertion surface is pressed through said annulus.

Preferably, said insertion surface provides a substantially constant size portion adjacent the distal end of said support portion.

The insertion surface may be conical or frustoconical in shape, but preferably is convexly rounded in shape, i.e. shaped like one end of an acorn.

Preferably, the support surface is shaped to engage the interior of a heart valve to be inserted by the tool; most preferably, the support surface is shaped to engage with all of the inner circumference of stent of a heart valve.

The body portion may be formed in any number of parts, but preferably is formed in either three or six parts. Preferably also, these parts are arranged to interengage when the body portion is in the first configuration. The parts of the body portion may be linked together by connecting means when the body portion is in the second configuration, to prevent the parts accidentally dropping into the patient. The connecting means can be any suitable means, e.g. ties, sutures, flexible strips, hinge strips.

The parts of the body portion can form the whole of the body portion, but preferably the tool further includes a central support around which the parts of the body portion are arranged, and with which the parts engage when the body portion is in the first configuration; to reconfigure the body portion to the second configuration, the central support is disengaged from the parts of the body portion.

The central support may be any of a wide range of shapes, e.g. generally cylindrical be frustoconical. Preferably, some of the parts of the body portion are formed with means for releasably engaging with the central support when the body portion is in the first configuration.

The tool may also include a handle releasably engageable with the proximal end of the central support.

The central support may also be formed with means for engaging one or more of the struts of a heart valve mounted on the insertion tool, to prevent relative rotation between the valve and the tool.

The present invention also provides a method of using the insertion tool as described above, said method including the steps of:

said method including the steps of:
configuring the body portion of the tool to said first configuration;
mounting a heart valve upon the insertion tool;
using said tool carrying said valve to elastically expand the annulus into which the valve is to be fitted;
using said tool to position said valve correctly within the annulus, such that said annulus contracts elastically around the perimeter of said valve;
reconfiguring the body portion to said second configuration and withdrawing said tool, leaving the valve positioned in the annulus.

Preferably, the heart valve is a rigid heart valve with a peripheral stent, and is mounted on the body portion in the first configuration of the body portion such that the inner margin of the stent is seated on, and fully supported by, the support portion of the body portion.

### BRIEF DESCRIPTION OF DRAWINGS

By way of example only, preferred embodiments of the present invention are described in detail, with reference to the accompanying drawings, in which:

FIG. 1 is a side view of a first embodiment of the insertion tool of the present invention, holding a valve in place ready for insertion;

FIG. 2 is a longitudinal cross-section through the insertion tool and valve shown in FIG. 1;

FIG. 3 is a plan view taken in the direction of arrow A of FIG. 1;

FIG. 4 is a plan view similar to that of FIG. 3, but showing a second embodiment of the invention;

FIG. 5 is a plan view of a third embodiment of the invention;

FIG. 6 is a simplified longitudinal section through the embodiment of FIG. 5;

FIG. 7 is a plan view in the direction of arrow C of FIG. 6;

FIG. 8 is a side view of a fourth embodiment of the insertion tool of the present invention, holding a valve in place ready for insertion;

FIG. 9 is a side view of part of the insertion tool of FIG. 8, with the valve removed, and with some of the components of the body portion removed, so that the shape of the components can be seen;

FIG. 10 is a plan view taken in the direction of arrow E in FIG. 10;

FIG. 11 is a plan view similar to FIG. 10, but of a variant design; and

FIG. 12 is a longitudinal section through the tool as shown in FIG. 10.

### BEST MODE FOR CARRYING OUT THE INVENTION

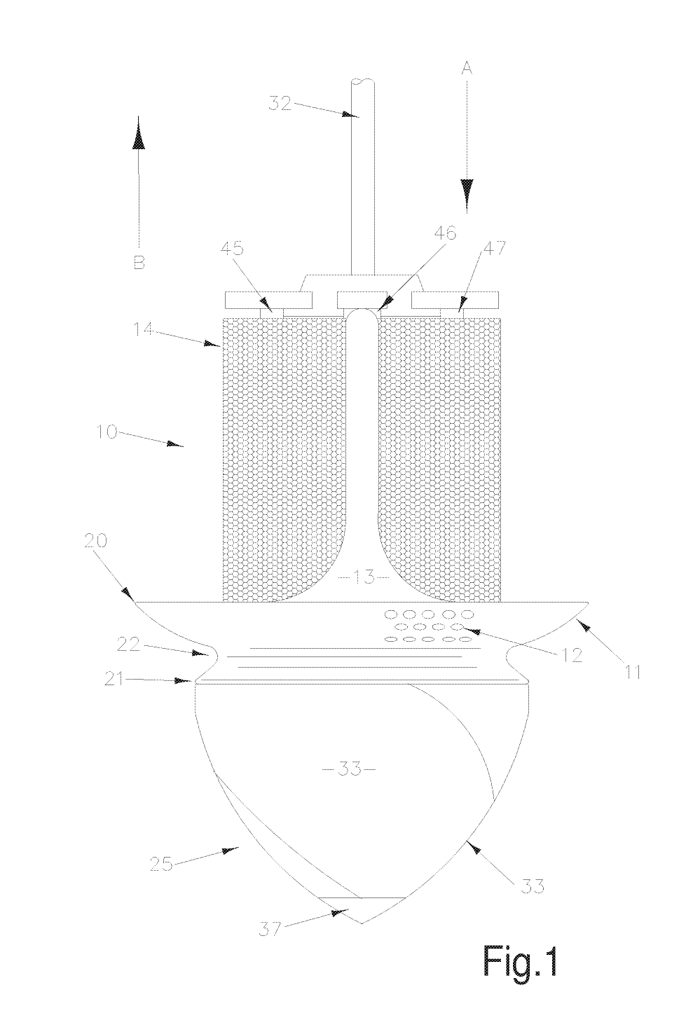

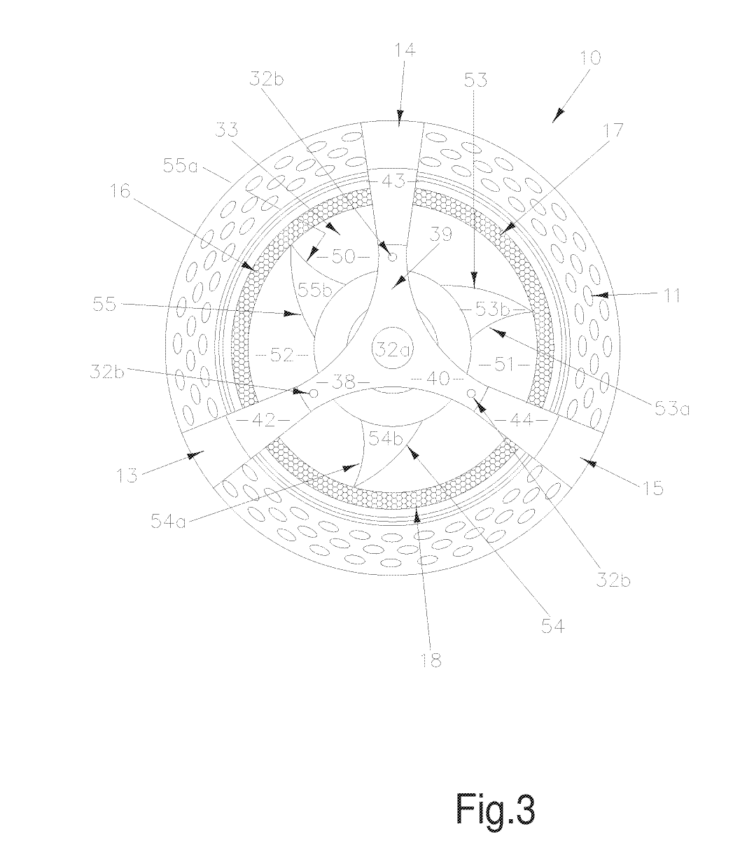

Referring to the drawings, FIG. 1 shows a stented prosthetic heart valve 10 in accordance with New Zealand Patent No. 527025, positioned on an insertion tool in accordance with a first embodiment of the present invention. The valve 10 includes a rigid peripheral stent 11 which is apertured around the whole of its circumference by apertures 12 (see FIG. 3) to allow suturing through the stent. Three equidistantly spaced struts 13, 14, 15 project from one face of the stent 11. Flaps of chainmail are secured between the stent 11 and the struts 13/14, 14/15, 15/13 to form valve leaflets 16, 17, 18 respectively.

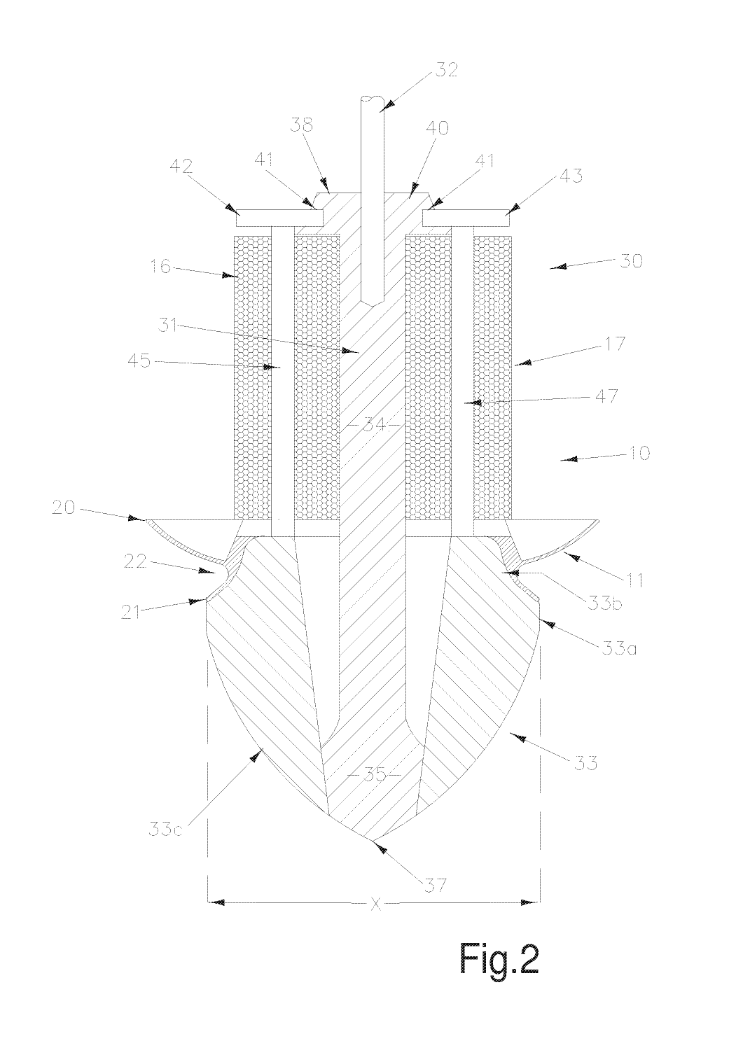

As shown in FIGS. 1 and 2, the exterior shape of the stent 11 provides a larger diameter end 20, to which the struts 13, 14, 15 are secured, and a smaller diameter end 21 from which one end of the insertion tool 25 projects. Between the ends 20 and 21, the exterior shape of the stent 11 curves inwards to provide a “waist” 22 which is smaller in diameter than the smaller end 21. The waist 22 provides a seating for the annulus when the prosthetic valve is correctly located.

In the position shown in the Figures the valve is open, i.e. the leaflets 16, 17, 18 are apart, to allow blood to flow through the valve. The chainmail of which the leaflets are made is flexible, so that the leaflets can flex towards each other to close off the valve and prevent flow of blood back through the valve.

The perforations 12 in the stent and the chainmail structure of the leaflets both encourage rapid endothelialisation; to prevent blood from passing through the chainmail of the leaflets until endothelialisation occurs, the valve may be fitted to the patient with an initial sealing coat of heat-treated blood (taken from the patient) or an initial sealing coat of biodegradable plastics material which breaks down in situ as endothelialisation occurs.

The above described valve is rigid (apart from the leaflets), and thus cannot be compressed to allow the valve to expand against the annulus to achieve a snug fit. Instead, the tool of the present invention is used to gently expand the annulus elastically and then insert the valve stent in the correct position relative to the annulus, so that the annulus can contract elastically around the valve stent, giving a snug, blood-tight fit between the annulus and the stent.

In the first embodiment, shown in FIGS. 1-3, the insertion tool 30 consists of three parts—a central support 31, a handle 32, and a body portion 33. The three parts are detachable from each other, as hereinafter described.

The central support 31 consists of a stem 34, which is generally cylindrical except at each end. Adjacent the distal edge of the tool 30, (i.e. the end of the tool which engages the annulus in use) the stem 34 is formed integrally with a core 35 which is enlarged in diameter and then narrows to a point 37.

The end of the stem 34 remote from the point 37 is formed with three equidistantly spaced arms 38, 39, 40 (see FIG. 3). Each of the arms 38, 39, 40, is slotted with slots 41 (see FIG. 2) to releasably engage cross pieces 42, 43, 44 which are formed on the ends of three equidistantly spaced uprights 45, 46, 47, formed integrally with the body portion 33.

The interior of each slot 41 is sized to be a sliding fit over the exterior of the corresponding crosspiece 42, 43, 44, so that the central support can be quickly and easily disengaged from the body portion by rotating the central support either clockwise or counterclockwise, to rotate the slots 41 clear of the cross pieces 42, 43, 44. Rotation of the central support, and general manipulation of the insertion tool, is carried out using the handle 32 in the form of a rod which is screw-threaded at one end to engage with a screw threaded socket 32a formed in the centre of the central support between the arms 38, 39, 40.

The uprights 45, 46, 47, are dimensioned and arranged to correspond in position to the struts 13, 14, 15 of the heart valve 10, and the exterior surface of each of the uprights is formed with one or more projections (not shown) to releasably engage the corresponding strut.

The body portion 33 provides a support portion 33b, which supports the valve 10 as shown in FIGS. 1 and 2, and an insertion surface 33c.

The body portion 33 of the insertion tool is formed in three separable parts 50, 51, 52, which are divided by curved edges 53, 53a, 54, 54a, 55, 55a, with cutout portions 53b, 54b, 55b, between each pair of adjacent curved edges 53/53a, 54/54a, 55/55a, to give clearance between the adjacent parts when the body portion 33 is collapsed inwards as hereinafter described. The exterior surface of the insertion surface 33c of the body portion 33 forms a convexly rounded, generally acorn shaped component which tapers outwards from the lower end 37 to a diameter X (FIG. 2) which is sized to provide a secure positive seating for the edge 21 of the prosthetic valve 10.

It will be noted that the body portion 33 has a portion 33a immediately below the edge 21 which is of substantially constant diameter.

The remaining part of the body portion 33 forms the support portion 33b, which is dimensioned to support the interior of the prosthetic valve 10, as shown in FIG. 2. It will be appreciated that the shape of the support portion 33b is varied as necessary, to suit the shape of the valve with which the tool is used.

It follows from the above that, when the prosthetic heart valve 10 is in position on the insertion tool 30 as shown in the drawings, the valve is supported in the correct position for insertion, with each of the valve struts 13, 14, 15 positively located on the insertion tool and the support portion 33b engaged with the interior of the stent 11 of the valve such that the tool can be used to manoeuvre the valve into position. Once the valve 10 is positioned as shown in the drawings, the engagement between each of the struts 13, 14, 15 and the corresponding uprights 45, 46, 47 prevents relative rotation between the insertion tool and the valve, and the valve cannot slide in the direction of arrow B (FIG. 1) because of the engagement between the end of each strut 13, 14, 15 and the corresponding crosspiece 42, 43, 44.

The above described insertion tool is used as follows—the patient's natural heart valve is prepared for the insertion of the prosthetic valve by the removal of the damaged heart leaflets and any other damaged material in the region of the annulus. Next, the surgeon estimates the size of the annulus using a sizer shaped like the insertion tool but made as a one-piece solid; a range of sizers is available, with a corresponding range of different sizes of valves and matching insertion tools.

Once the size of the annulus has been ascertained, the surgeon selects the corresponding size of valve 10 and insertion tool 30, and positions the valve on the insertion tool, as shown in the drawings; the valves and corresponding insertion tools may be supplied already positioned for insertion.

The surgeon then inserts three equidistantly spaced guidance stitches, each of which extends from the annulus to one of the apertures 32b on the tool. The surgeon uses the handle 32 to manipulate the insertion tool 30 carrying the valve 10, pushing the end 37 of the insertion tool through the annulus until the annulus engages the waist 22 of the stent 11. The provision of the substantially uniform diameter portion 33a of the body portion 33 assists in correctly locating the valve on the annulus by providing the surgeon with an improved tactile feedback as the valve is being fitted.

The acorn shape of the insertion surface 33c of the insertion tool gently stretches the annulus during insertion, and expands the annulus elastically until the annulus passes over the end 21 of the stent 11 and then contracts elastically to engage around the waist 22. Once the annulus is securely engaged with the stent 11 in this way, the valve 10 is securely and correctly positioned, and the guide sutures may be either tied off or removed.

The insertion tool 30 must now be disengaged from the installed valve 10. This is done by using the rod 32 to rotate the central support 31 clockwise or counterclockwise, sufficient to disengage the slots 41 from the cross pieces 42, 43, 44. The handle 32 and the central support 31 are then pulled away from the body portion 33, in the direction of arrow B in FIG. 1.

The tapered shape of the core 35 assists in the smooth and gentle withdrawal of the central support 31, without disturbing the positioning of the valve 10. Once the core 35 has been removed from the insertion tool, the three component parts 50, 51, 52, of the body portion 33 are no longer supported in the centre and can slide past each other along the curved edges 53, 53a, 54, 54a, 55, 55a, to fold down to an overall exterior dimension substantially smaller than the interior of the stent 11, so that the body portion 33 can be withdrawn from the patient by grasping the cross pieces 42, 43, 44 and extracting the body portion 33 through the valve in the direction of arrow B in FIG. 1.

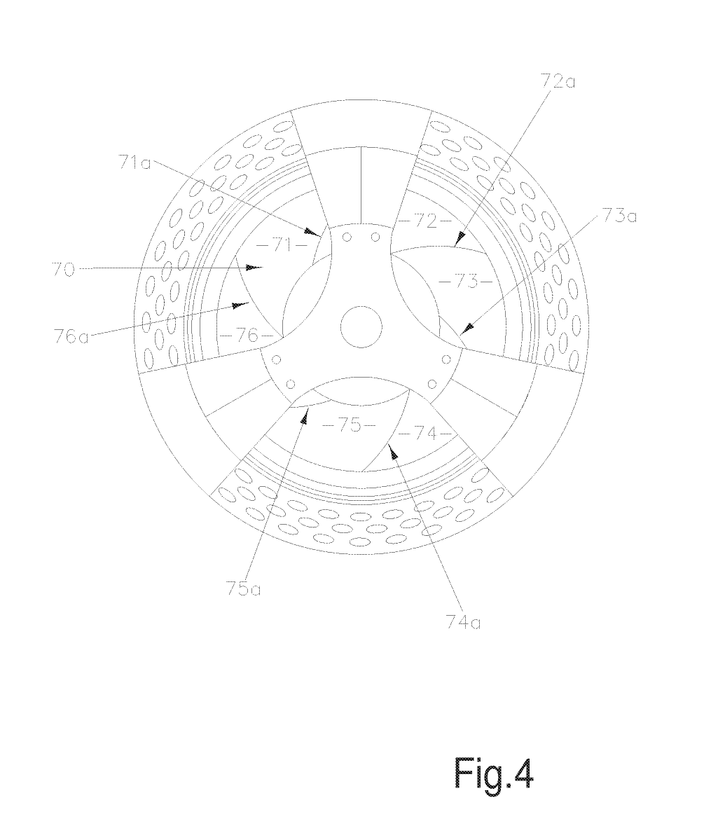

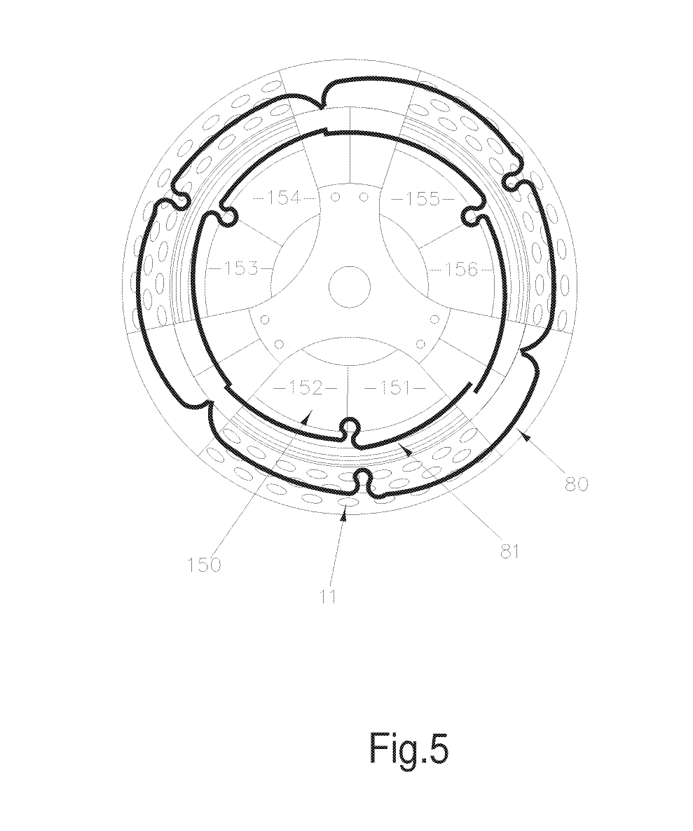

FIG. 4 shows a second embodiment of the present invention, which is identical to the first embodiment except that the body portion 70 of the tool is divided into six portions 71, 72, 73, 74, 75, 76, separated from each other by curved edges 71a, 72a, 73a, 74a, 75a, 76a. Dividing the body portion into six portions rather than three makes it easier for the body portion to collapse into a smaller space when the central support 31 is withdrawn. However, having so many segments does increase the risk of one or more of the segments dropping down through the annulus and having to be retrieved. This could be prevented by linking alternate portions of the body portion 70 by connecting means such as ties 80, 81 as shown the embodiment described with reference to FIG. 5.

In the embodiment shown in FIG. 5, the body portion 150 is divided into six straight sided portions 151, 152, 153, 154, 155, 156. The tie 80 is secured to alternate portions of the body portion and the tie 81 is secured to the remaining portions of the body portion. The portions can then be retrieved by drawing the ties in the direction of arrow B of the FIG. 1 once the central support 31 has been withdrawn. The ties 80, 81, may be any suitable connecting means, e.g. sutures, flexible strips, hinge strips.

It is envisaged that removal may be facilitated by initially pushing one set of alternate body portions (linked by one of the ties 80, 81) in the direction of Arrow A, then withdrawing the other set of body portions by drawing the corresponding tie in the direction of Arrow B, then finally withdrawing said one set in the direction of Arrow B.

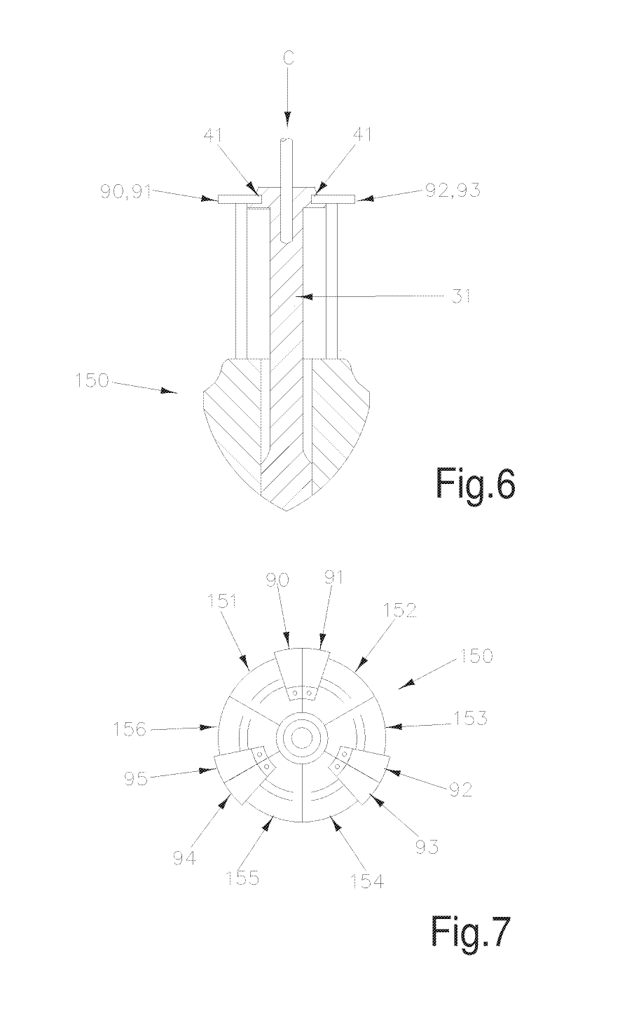

As shown in FIGS. 6 and 7, the six portions of the body portion 150 are assembled to form a body portion of substantially the same shape as the three-part body portion 33. The six portions are each formed with cross pieces 90, 91, 92, 93, 94, 95, (as shown in FIG. 7) and these cross pieces are arranged in pairs to engage in the slots 41 as shown in FIG. 6.

It would also be feasible to construct the insertion tool as a multi piece body portion without a central support, with the pieces of the body portion being releasably clamped together to support the valve in the manner described above while the valve is being inserted and then disassembled into the component parts to deconstruct the insertion tool for easy removal once the valve has been fitted. A tool of this type preferably would have the portions of the body portion linked by connecting means as described with reference to FIG. 5, and could be withdrawn from the valve after use by using either of the techniques described with reference to FIGS. 5-7.

The above described embodiment and the embodiments described with reference to FIGS. 4-7 are used in the same manner as the embodiment described with reference to FIGS. 1-3.

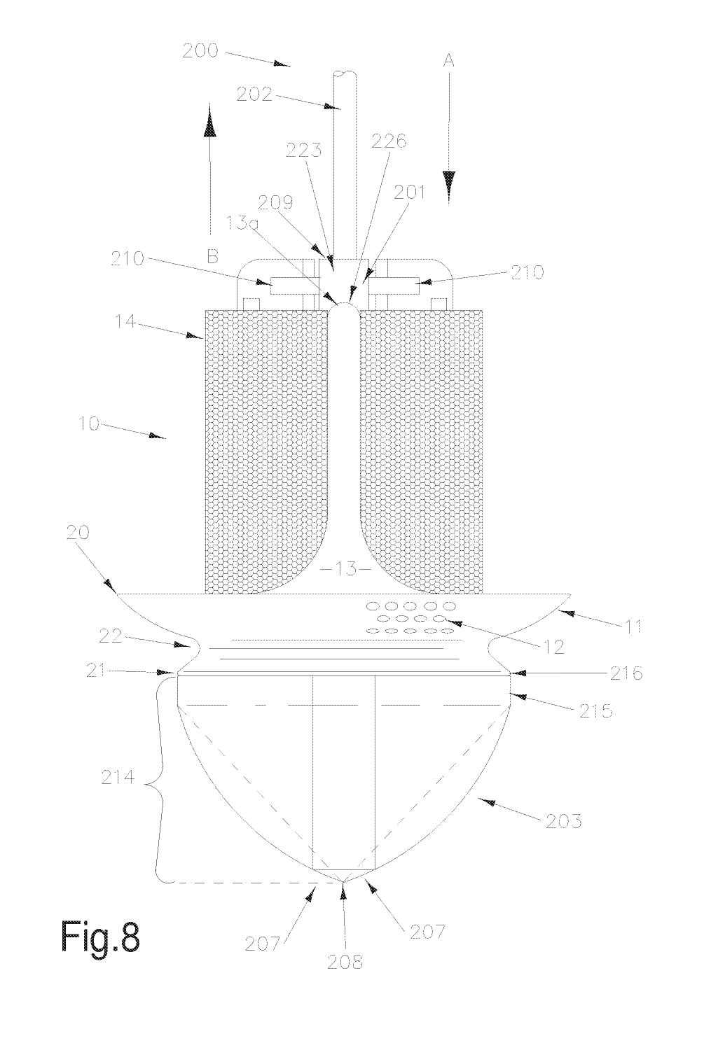

A fourth embodiment of the invention is shown in FIGS. 8-12.

Referring to FIG. 8, a stented prosthetic heart valve 10 in accordance with NZ patent number 527025 is shown engaged with an insertion tool 200.

The insertion tool 200 consists of three parts—a central support 201, a handle 202, and a body portion 203. The handle 202 is partially shown only in FIG. 8, and may be in any convenient form; one end of the handle 202 is designed to engage a socket 204 formed in one end of the central support 201, such that the handle can be used to manipulate the central support.

The central support 201 is designed to provide a “keystone” for the tool when assembled (i.e. in the first configuration) and is circular in cross-section along a majority of its length. A first portion 205 of the central support is formed as a tapered cylinder which gradually decreases in diameter from a shoulder 206 formed at approximately the midpoint of the length of the support 201, to one end of the support 201, which is formed into a shallow conical shape with sides 207 inclined towards a tip 208.

The end of the central support 201 opposite to the tip 208 is formed in a smooth concave curve to terminate in a three-armed end 209, with three equidistantly spaced arms 210.

The socket 204 is formed in the centre of the end 209.

Each of the three arms 210 is dimensioned and positioned to be an easy sliding fit within a corresponding slot 211 formed adjacent one end of each of three of the parts of the body portion, as hereinafter described.

The body portion 203 is formed from six completely separate parts—three identical shorter segments 212 and three identical longer segments 213. To form the body portion, the parts 212 and 213 are arranged symmetrically around the central support 201, with the parts alternating, i.e. each part 212 is arranged with a part 213 in contact with each edge.

When the parts 212/213 are assembled round the central support 201 to form the completed body portion 203, the body portion and central support together are designed to hold the valve 10 accurately and securely in a preselected position and orientation, so that a surgeon can position the valve 10 exactly. Once the valve 10 is correctly positioned, the insertion tool 200 is dismantled as described below, so that it can be withdrawn from the patient without in any way disturbing the valve's position.

To hold the valve 10 correctly, the body portion 203 assembled around the central support 201 provides an insertion surface 214 (FIG. 8 only) which in use lies distally of the valve 10 and which is shaped rather like an acorn—it provides a smooth convexly rounded, curved surface which decreases steadily in cross-sectional diameter from a parallel sided portion 215 down to the tip 208. At the end of the parallel sided portion 215 furthest from the tip 208, a shoulder 216 is formed to provide a seating for the valve 10; the exterior diameter of the shoulder 216 is slightly greater than the interior diameter of the smaller diameter end 21 of the stent 11.

Above the shoulder 216 the remaining part of the body portion 203 provides a support portion in the form of a smoothly tapering surface, the curve of which matches the curve of the interior of the stent 11, so that the valve 10 is provided with firm support all around the interior of the stent 11.

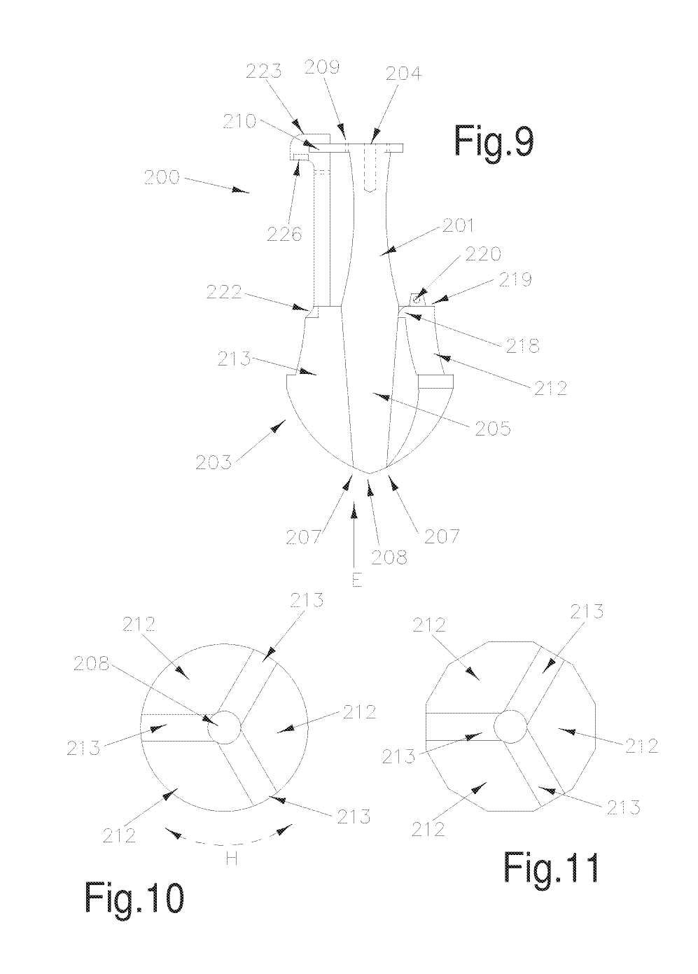

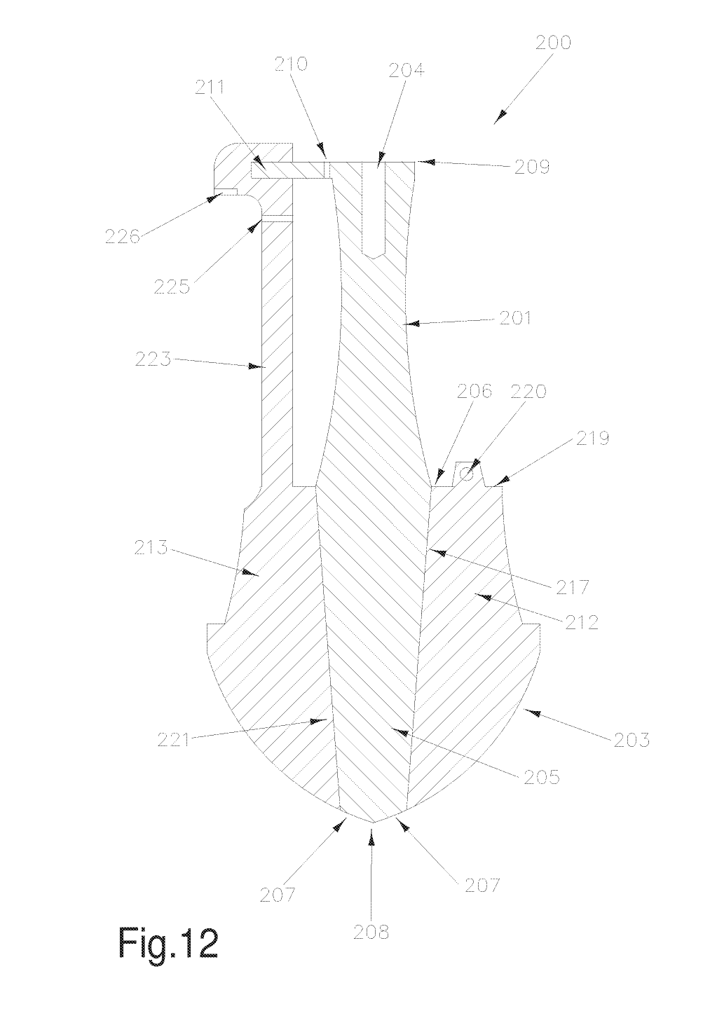

As shown in FIGS. 9 and 12, each of the three shorter segments 212 is formed as a segment of the above described shape, with a central tapered cavity 217, to permit each of the segments 212 to engage smoothly with the exterior of the central support 201. Each of the shorter segments 212 also is formed with a small projecting flange 218 (FIG. 10 only) at each side of the segment 212, at the edge 219 of the segment furthest from the tip 208. An eyelet 220 is formed in the centre of each edge 219, extending outwardly from the edge; the eyelet 220 receives a suture in use, as described below.

Each of the longer segments 213 is formed as a smaller segment of the shape of the assembled body portion 203 and, like the shorter segments 212, is formed with a central tapered cavity 221 to engage smoothly with the exterior of the central support 201. Each of the longer segments 213 also is formed with a pair of shoulders 222 (FIG. 9 only) which are dimensioned and positioned to engage the flanges 218 on each of the adjacent shorter segments 212 when the segments are assembled into the body portion 203.

The end of each of the longer segments 213 furthest from the tip 208 also is formed with an arm 223 the free end of which is formed with a slot 211 which is positioned and dimensioned to receive one of the arms 210 as a sliding fit therein.

Each of the arms 223 is apertured with an aperture 225 a short distance below the slot 211, so that a connecting means such as a suture (or other suitable connecting means, as described above) can be threaded through in use.

The above described tool is used as follows—first, a suture is threaded through each of the eyelets 220 on the shorter segments 212, and then threaded through the apertures 225 in each of the longer segments 213; this secures all of the segments together, spaced apart along the suture.

Next, the body portion 203 is assembled around the central support 201 by arranging the shorter segments 212 alternately with the longer segments 213, around the central support 201, and engaging each of the slots 211 with one of the arms 210.

The engagement between the flanges 218 and the shoulders 222 positions the longer and shorter segments correctly with respect to each other and to the central support 201.

The valve 10 to be inserted in a patient is then positioned as shown in FIG. 8, with the end 21 of the stent 11 seated on the shoulder 216 of the tool, and with the free end of each of the valve struts (only one of which, 13a, is visible) engaged with a curved recess 226 formed on the outer surface of the corresponding arm 223. This engagement prevents the valve 10 from rotating relative to the tool during insertion.

Optionally, to orient the valve 10 correctly in the annulus in which it is to be located, three guide sutures may be placed between the walls of the annulus and the apertures 12 in the peripheral stent 11. However, guide sutures need not be used.

The handle 202 is then inserted in the tool as described above, and the tool carrying the valve is lowered into the annulus. The shape of the insertion surface 214 of the body portion 203, which increases gradually in diameter towards the lower edge 21 of the stent 11, gently and elastically expands the wall of the annulus as the tool is inserted. The straight sided portion 215 of the body portion 203 provides the surgeon using the tool with a tactile indication that the lower edge of the stent 11 has nearly been reached, and the surgeon can then feel that a further small movement of the tool allows the annulus to contract around the waist 22 of the stent 11. The valve 10 is now securely and correctly positioned; the guide sutures, if used, may be tied off or removed.

The tool 200 is now disassembled to allow the tool to be removed. First, the handle 202 is used to rotate the central support 201 as indicated by arrows H in FIG. 10, to disengage the arms 210 from the corresponding slots 211. The handle 202 is then used to remove the central support 201 from the tool. This allows the parts 212/213 to disengage from each other, and the connecting suture is used to remove these parts, one at a time, from the interior of the valve.

In all of the above described embodiments, the insertion surface (33c, 214) has been described as having a rounded, convex shape. However, the rounding is not essential—the insertion surface could be formed with straight sides, (i.e. as a frustoconical portion) if preferred; this is illustrated in broken lines in FIG. 8. Further, the insertion portion need not be circular in cross-section, but could be polygonal, as illustrated in FIG. 11.

It will be appreciated that the above described insertion tool is at all times positively engaged with the valve to be inserted, so that the valve can be manipulated easily and accurately.

The insertion tool provides greatly improved tactile feedback to the surgeon, so that the surgeon can judge whether or not the valve stent is correctly seated in the annulus. A further advantage is that suturing is reduced or eliminated, reducing the overall time required for fitting the valve.

When a heart valve needs to be replaced by a prosthetic valve, it is advantageous for the patient if the prosthetic valve can provide at least the same blood flow capacity as the original valve. Thus, prosthetic valves which, by the nature of their design, reduce the diameter of the passage available for blood flow, are not optimal. The widely used cloth sewing cuff provided on many existing prosthetic valves requires a degree of under sizing for a correct fit, and this tends to reduce the passage diameter. In contrast, a valve fitted using the insertion tool of the present invention maximises the valve orifice.

A further advantage of the tool and method of the present invention is that they may be used in combination with any of a wide range of prosthetic valves, whether the valves are designed as rigid valves or as collapsible valves.

The tool of the present invention also could be used to assist in the installation of a trans-catheter valve. To install such a valve, the valve is collapsed and mounted on a catheter, and then expanded at the position in which it is to be inserted. The tool of the present invention could be used to expand the collapsed valve and to ensure that it is seated correctly.

The tool may be made from any of a wide range of medically acceptable materials.

1. An insertion tool for a heart valve, said tool including
a solid body portion which provides a surface of a first size and shape in a first configuration, and which forms two or more parts each of which is of a lesser size than said first size and shape, in a second configuration;
wherein, in said first configuration said body portion provides:
a support portion for supporting thereon a valve to be inserted by said tool; and
an insertion surface arranged to protrude distally from said support portion;
and wherein said insertion surface is tapered from a small cross-sectional size distally of said support portion to a greater cross-sectional size proximally to said support portion, such that in use said insertion surface can gently and elastically expand the periphery of an annulus into which said valve is to be fitted, as said insertion surface is pressed through said annulus.

2. The insertion tool as claimed in claim 1, wherein said insertion surface provides a substantially constant size portion adjacent the distal end of said support portion.

3. The insertion tool as claimed in claim 1 or claim 2, wherein said insertion surface is conical or frustoconical in shape.

4. The insertion tool as claimed in claim 1 or claim 2, wherein said insertion surface is convexly rounded in shape.

5. The insertion tool as claimed in claim 1, wherein said support surface is shaped to engage the interior of a heart valve to be inserted by said tool.

6. The insertion tool as claimed in claim 5, wherein said support surface is shaped so as to engage with all of the inner circumference of a stent of a heart valve.

7. The insertion tool as claimed in claim 1 or claim 5, wherein said body portion is formed in three parts.

8. The insertion tool as claimed in claim 1 or claim 5, wherein said body portion is formed in six parts.

9. The insertion tool as claimed in claim 1, wherein the parts of said body portion inter-engage when said body portion is in said first configuration.

10. The insertion tool as claimed in claim 1, wherein the parts of said body portion are linked together by connecting means when said body portion is in said second configuration.

11. The insertion tool as claimed in claim 1, wherein said tool further includes a central support around which the parts of the body portion are arranged.

12. The insertion tool as claimed in claim 11, wherein said central support engages with the parts of the body portion when the body portion is in said first configuration, and said central support is disengaged from the parts of the body portion when the body portion is in said second configuration.

13. The insertion tool as claimed in claim 12, wherein said central support is generally cylindrical, with a large diameter portion at each end.

14. The insertion tool as claimed in claim 13, wherein said central support contacts the parts of the body portion at each of said larger diameter portions.

15. The insertion tool as claimed in claim 11, wherein said central support provides a frustoconical portion for contacting said parts of said body portion.

16. The insertion tool as claimed claim 11, wherein at least some of the parts of the body portion are formed with means for releasably engaging with said central support when said body portion is in said first configuration.

17. The insertion tool as claimed in claim 11, wherein the tool further includes a handle releasably engageable with the proximal end of the central support.

18. The insertion tool as claimed in claim 11, wherein said central support is formed with means for engaging one or more valve struts of a heart valve, so as to prevent relative rotation between said valve and said tool when the valve is mounted on said tool.

19. The insertion tool as claimed in claim 1, wherein the sides of the parts of the body portion which contact each other, are curved.

20. The insertion tool as claimed in any one of claims 1-claim 1, wherein the sides of the parts of the body portion which contact each other, are flat.

21. The combination of an insertion tool as claimed in claim 1, and a heart valve.

22. The combination as claimed in claim 21, wherein said heart valve is a rigid heart valve which includes a peripheral stent.

23. A method of using the insertion tool as claimed in claim 1 or claim 11, said method including the steps of:
configuring the body portion of the tool to said first configuration;
mounting a heart valve upon the insertion tool;
using said tool carrying said valve to elastically expand the annulus into which the valve is to be fitted;
using said tool to position said valve correctly within the annulus, such that said annulus contracts elastically around the perimeter of said valve;
reconfiguring the body portion to said second configuration and withdrawing said tool, leaving the valve positioned in the annulus.

24. The method as claimed in claim 23, wherein said heart valve is a rigid valve having a peripheral stent, and said valve is mounted upon the body portion in said first configuration such that the inner margin of said stent is seated on, and fully supported by, the support portion of said body portion.
